(19) United States
(12) Patent Application Publication
(10) Pub. No.: US 2003/0079241 A1
(43) Pub. Date: Apr. 24, 2003

(54) Premalignant, serially-transplantable breast tissue lines and uses thereof

(76) Inventors: Robert D. Cardiff, Davis, CA (US); Carol L. MacLeod, San Diego, CA (US)

Correspondence Address: Benjamin Aaron Adler, ADLER & ASSOCIATES, 8011 Candle Lane, Houston, TX, 77071, US

(21) Appl. No.: 10/162,181

(22) Filed: Jun. 4, 2002

Related U.S. Application Data
(60) Provisional application No. 60/295,654, filed on Jun. 4, 2001.

(51) Int. Cl. A01K 67/033; C12N 5/06

(52) U.S. Cl. 800/10; 800/18; 435/354

(57) ABSTRACT

The present invention provides premalignant, serially-transplantable, hyperplastic outgrowth breast tissue lines and methods of isolating these tissue lines from mice expressing a transgene which genetically predisposes said mice to develop breast cancer. This is accomplished by implanting cells from said foci both subcutaneously and in the inguinal mammary fat pads of syngeneic, non-transgenic mice; identifying foci which grow in said fat pads but not subcutaneously as premalignant, hyperplastic outgrowths; and, establishing clonal serially-transplantable breast tissue lines from said outgrowths. Such premalignant, serially-transplantable, hyperplastic outgrowth breast tissue lines are useful for screening chemotherapeutic and chemopreventative agents as well as for identifying biomarkers of mammary preneoplasia.

### CROSS-REFERENCE TO RELATED APPLICATION

This non-provisional patent application claims benefit of provisional patent application U.S. Serial No. 60/295,564, filed Jun. 4, 2001, now abandoned.

### BACKGROUND OF THE INVENTION

1. Field of the Invention

The present invention relates generally to the field of breast cancer oncogenesis. Most specifically, the present invention relates to novel, stable, premalignant, serially-transplantable, hyperplastic outgrowth (HPO) breast tissue lines that have a predictable rate of tumor formation and predictable morphology and uses thereof.

2. Description of the Related Art

A more complete understanding of the biology of hyperplastic epithelial lesions is clearly essential for improving intervention and treatment of human breast disease. The current method for diagnosing breast disease is based upon histopathology (1), while prognosis and decisions concerning treatment rely on epidemiological studies that correlate specific histopathological findings with clinical outcomes (2, 3). The lack of knowledge regarding the malignant potential of hyperplastic lesions continues to hamper accurate prediction of clinical outcomes. The lack of certainty regarding which cells will become malignant impedes the development of treatments designed to target premalignant cells. However, human investigations remain limited to retrospective study designs that provide only relative risk statistics. Therefore, it is critical to augment human retrospective studies with detailed analyses of premalignant lesions that include both histology and biological experiments testing growth behavior (4-7).

The mouse has emerged as the primary animal model system for human breast development and the analysis of their disorders. The murine mammary gland is similar in structure and function to the human (8, 9). Recent advances have permitted the creation of mice that have a variety of distinct mammary disorders. The unique patterns observed in the transgenic mammary gland are comparable to hyperplasias and adenocarcinomas found in the human mammary gland. Even experienced pathologists have difficulty distinguishing between lesions from the two species (8, 9). The morphological similarities reinforce the utility of mouse models for understanding human breast cancer. In some transgenic strains, tumors develop rapidly and undergo reproducible metastasis. The early events prior to the onset of malignancy have not been systematically assessed or characterized in any transgenic mouse model. Genetically engineered mice have emerged as powerful tools for investigating neoplastic progression within an intact array of host factors that are not present in in vitro models (10-16).

To address the need for a more comprehensive and uniform analysis of mammary lesions in transgenic models, a panel of expert pathologists was convened by the NCI at Annapolis (6). The panel recommended that diagnostic nomenclature of transgenic lesions in mice be based on similar morphological, immunohistochemical and biological criteria so that direct comparisons among mouse models and with humans would be possible. The use of the term Mammary Intraepithelial Neoplasia (MIN) was proposed for premalignant lesions associated with cancer, with the provision that their premalignant properties be confirmed using classical mammary gland transplantation experiments (6).

While multi-step transgenic models of tumorigenesis and progression are particularly useful in the study of premalignant lesions, characterization of early proliferative lesions is not yet complete. A recent literature review of the histopathological and biological properties of premalignant mammary lesions in transgenic mice identified 70 papers describing potential premalignant lesions (5). Few contain mammary transplantation experiments and none fulfill the rigorous requirements proposed by the Pathology Panel for the diagnosis of mammary intraepithelial neoplasia (6). Thus, currently no mouse model has been clearly established as bearing lesions that have been proven to be pre-malignant (20).

The lack of knowledge regarding the biological fate of hyperplastic lesions hampers accurate prediction of clinical outcomes. Further, the lack of certainty regarding which cells will become malignant makes it difficult to design treatments that target premalignant cells. It has therefore become increasingly clear that an understanding of the biology of preneoplasia is essential for improving both prognosis and treatment regimes. An in-depth analysis of preneoplastic lesions in models of mammary cancer that include biological experiments, is an essential step in understanding the rules governing the transformation of normal cells to malignancy (21-25).

The prior art is deficient because of the lack of stable, premalignant, serially-transplantable breast tissue lines that develop tumors at a predictable rate with a predictable morphology for the study of molecular mechanism in early proliferative lesions. It is also deficient in useful models for preclinical screening of chemopreventative and chemotherapeutic agents. The present invention fulfills this longstanding need and desire in the art.

### SUMMARY OF THE INVENTION

The present invention provided methods of isolating premalignant, serially-transplantable hyperplastic outgrowth (HPO) breast tissue lines from mouse mammary gland tissue occurring distal from the nipple (zones 3 and 4) and proximal to the nipple (zone 1). The present invention also includes premalignant, serially-transplantable hyperplastic outgrowth (HPO) breast tissue lines isolated by these methods such as hyperplastic outgrowth (HPO) breast tissue lines A, B, C, D, and E from zone 4 of the mammary gland.

The instant invention further provides methods of using the hyperplastic outgrowth breast tissue lines disclosed herein. These cell lines can be used in various screening methods to screen for compounds such as new chemopreventive agents against breast cancer, chemotherapy agents which block metastasis, and biomarkers characteristic of premalignant or malignant breast cells.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows an inguinal mammary fat pad divided into 4 topographical zones. The lymph node serves as a convenient guide, dividing the rest of the mammary fat pad roughly into halves. Note the solid invasive lesion in zone 1. The two lower images are enlarged areas of the whole mount showing a cystic lesion (Arrow C) and a solid lesion (Arrow S). Early lesions start at the terminal ducts that may have thickening of the duct lining (Arrow D). One can compare the thickness of this duct with that of a normal duct (Arrow N).

FIG. 2 shows Mammary Intraepithelial Neoplasia/Tumor comparisons from photo images of mammary intraepithelial neoplasia and tumor in nine-week old virgin female mice carrying the PyV-mT transgene. The top row illustrates the cytological and organizational detail of PyV-mT mammary intraepithelial neoplasia and tumor. Note that mammary intraepithelial neoplasia cells are confined within a basement membrane but are multi-layered and form ill-defined glands whereas tumor cells are not surrounded by a basement membrane. The cycling Ki-67 positive cells in mammary intraepithelial neoplasia lesions tend to be concentrated at the periphery. This expression pattern corresponds with the distribution of cells that are most highly positive for PyV-mT RNA/antigen. Note that the tumor cells in sections identified with PyV-mT RNA probe (blue staining detects the probe) and Ki-67 surround a nerve fiber (arrows). Tumors have a more random PyV-mT RNA/protein and Ki-67 distribution pattern. The row of images stained for ER and PgR demonstrate that the mammary intraepithelial neoplasia and tumor cells contain identifiable ER antigen within the nuclei. The ER pattern is variable with patches of ER negative cells (arrow). Some ductal cells stain for PgR (arrow). However, neither the mammary intraepithelial neoplasia nor tumors have detectable levels of PgR antigen.

FIG. 3 shows the Immunohistochemistry of PyV-mT mammary intraepithelial neoplasia/tumors. Photo images of prelactating mammary gland (column 1), PyV-mT MIN (column 2) and PyV-mT mammary tumor (column 3) show the patterns of laminin staining, cytokeratin 8 (CK-8), cytokeratin 14 (CK-14), smooth muscle actin (SMA), whey acidic protein (WAP) and osteopontin (OPN). Note that CK-8 stains all epithelial cells in the prelactating and hyperplastic mammary gland but is variable in tumors. CK-14 is largely limited to the basal myoepithelium in normal mammary gland tissue. However, CK-14 appears in all layers, including the luminal surface, of mammary intraepithelial neoplasia lesions and is randomly distributed in the tumor. In contrast, smooth muscle actin positive myoepithelium is absent in mammary intraepithelial neoplasia and mammary tumors. Smooth muscle actin positive cells remain only in the smooth muscles of the blood vessels and myoepithelium surrounding normal ducts. Both whey acidic protein and osteopontin positive cells are found in mammary intraepithelial neoplasia. However, whey acidic protein falls below detectable levels in tumors.

FIG. 4 shows the microvasculature of PyV-mT mammary intraepithelial neoplasia/tumors: Photo images of India Ink injected whole mounts (column 1) and comparable frozen sections stained for CD31 (column 2) illustrate the three and two dimensional distribution of vessels in prelactating FVB/N mammary gland, PyV-mT mammary intraepithelial neoplasia and PyV-mT mammary tumor. Note the relative increases in vessel diameter and increased irregular distribution of the vessels in the whole mount preparations (column 1). When seen in the two dimensional sections (column 2), the number of vessels decreases relative to the epithelial area in the mammary intraepithelial neoplasia and the tumors.

FIGS. 5A and 5B show hyperplastic growth in an intact and cleared mammary fat pad. FIG. 5A shows the primary outgrowth of premalignant tissue. Hyperplastic foci isolated from zones 3 and 4 of 8 week-old transgenic mammary glands were transplanted into gland-cleared fat pads of syngeneic non-transgenic mice. After 10-17 weeks, non-invasive hyperplastic outgrowths were observed in 64% (18/24) of recipient fat pads. Microscopic foci of solid, invasive tumors arose in continuity with the hyperplastic tissue (arrow), suggesting that these were premalignant tissues.

FIG. 5B shows primary outgrowth in Intact Fat Pad. A fragment of PyV-mT hyperplastic mammary gland tissue was transplanted into zone 4 of an intact FVB female fat pad. When the PyV-mT mammary tissue grew to meet the FVB mammary gland, both tissues stopped growing and the normal gland turned away from the transplant (inset). This experiment illustrates that the PyV-mT hyperplastic tissue has not gained total autonomy but still responds to normal tissue growth inhibition.

FIG. 6 shows a time course of the proportion of mice developing tumors from the implantation of each premalignant, serially-transplantable hyperplastic outgrowth breast tissue line.

### DETAILED DESCRIPTION OF THE INVENTION

One widely used transgenic model of mammary tumorigenesis expresses the Polyoma Virus middle-T antigen (PyV-mT) (12). Tumor induction is so rapid in Polyoma Virus middle-T antigen transgenic mice that some investigators have postulated that there is no intermediate stage of tumorigenesis, and transgene expression alone is sufficient for mammary epithelial cell transformation (17, 18). However, the presence of focal atypias within the PyV-mT mammary gland is consistent with a multi-step model in which additional molecular events are required (19). The in vivo growth properties of these early proliferative lesions had not previously been studied (5) and it has not been clear whether PyV-mT transgenic mice serve as a model for single-step or multi-step transformation (12-18).

The experiments described herein provide a comprehensive histopathological and biological analysis of early proliferative lesions found in this widely used mammary cancer model. It is the first, in a transgenic model, that completely fulfills the Pathology Panel criteria for diagnosing mammary intraepithelial neoplasia and it provides a baseline for comparison with premalignant lesions in other models and in humans (5). Furthermore, a set of 5 different stable premalignant, serially-transplantable, hyperplastic outgrowth (HPO) breast tissue lines were established that make premalignant mammary tissue from PyV-mT mice available for detailed molecular analyses. A number of other HPO lines, which were not selected for further experimentation, were nonetheless partially investigated and found to have similar characteristics as the five lines selected for intensive experimentation. These lines are amenable to direct experimental manipulation and will facilitate analyses that are needed to augment data from human retrospective studies. Distinct tumor incidence among these lines reveal that PyV-mT-induced mammary intraepithelial neoplasia, like early proliferative lesions seen in the human breast, are heterogeneous with respect to their malignant potential. These results provide comprehensive evidence that Polyoma Virus middle-T antigen mice have premalignant hyperplastic foci that require additional “hits” before malignancy is acquired. The data firmly establish the utility of the PyV-mT system for the study of multi-step progression of mammary lesions from normal through hyperplasia to malignancy and metastasis.

The instant invention provides a method of isolating premalignant serially-transplantable hyperplastic outgrowth (HPO) breast tissue lines from regions of mouse mammary gland distal from the nipple (zone 3 and 4). This is accomplished by isolating hyperplastic foci from Zones 3 and 4 of mammary glands in transgenic mice before tumor development has occurred (before 8 weeks of age in the case of PyV-mt transgenic mice). Cells from the foci are implanted into inguinal mammary fat pads of syngeneic, non-transgenic mice as well as subcutaneously. Cells that grow in the fat pads but not subcutaneously are premalignant, serially-transplantable hyperplastic outgrowths and are used to establish clonal tissue lines. The instant invention is also directed to the premalignant serially-transplantable hyperplastic outgrowth (HPO) breast tissue lines isolated in this manner. Examples of these lines include, but are not limited to, transplantable hyperplastic outgrowth (HPO) breast tissue lines A, B, C, D, and E from zone 4 of the mammary gland of 8 week old PyV-mt transgenic mice.

The instant invention also describes a method of isolating additional premalignant serially-transplantable hyperplastic outgrowth (HPO) breast tissue lines from tissue proximal to the nipples of mouse mammary glands (zone 1). This may be accomplished by isolating hyperplastic foci from Zone 1 of mammary glands in transgenic mice before tumor development (4 week of age in the case of PyV-mt transgenic mice). The foci are then implanted both subcutaneously and into the inguinal mammary fat pads of syngeneic, non-transgenic mice. Foci that grow in the fat pads but not subcutaneously are identified as premalignant, hyperplastic outgrowths and are used to establish serially-transplantable breast tissue lines. The instant invention is also directed to the premalignant serially-transplantable, hyperplastic outgrowth (HPO) breast tissue lines isolated from this method.

The instant invention also provides a method of using the above described premalignant serially-transplantable, hyperplastic outgrowth (HPO) breast tissue lines to screen for new chemopreventative agents against breast cancer. The serially-transplantable breast tissue lines are implanted into the inguinal mammary fat pads of syngeneic, non-transgenic recipient mice. Putative chemopreventative agents are administered to a subset of the implanted mice. A chemopreventative agent effective against breast cancer is identified when fewer malignant tumors are observed in the serially-transplantable breast tissue line-implanted mice receiving the agent compared to similar serially-transplantable breast tissue line-implanted mice which did not receive it.

Breast tissue lines A, B, C, D, and E from zone 4 all express high levels of estrogen receptor, which is also expressed in human premalignant breast cells. This is important for testing chemopreventive agents that rely on this receptor. Moreover, the effects of tamoxifen on the development of tumors in mice receiving bilateral implants of HPO line A were examined. None of the mice receiving tamoxifen developed tumors after 15 weeks, whereas all mice receiving only the control vehicle developed bilateral tumors. This result strongly supports the validity of the model for screening chemopreventive agents.

The instant invention also directed to a method of using the above described premalignant serially-transplantable, hyperplastic outgrowth (HPO) breast tissue lines to screen for new chemotherapy agents which block metastasis. The serially-transplantable breast tissue lines are implanted into the inguinal mammary fat pads of syngeneic, non-transgenic recipient mice. A putative agent are administered to a subset of the implanted mice. After a period of time, the lungs of the cell-line implanted mice are examined for the presence of metastatic tumors. If fewer metastatic tumors are observed in the lungs of the cell-line implanted mice receiving the agent compared to similar mice that did not receive said agent, the chemotherapeutic agent is effective in blocking metastasis.

The instant invention is also directed to a method of identifying biomarkers for premalignant breast cells by comparing expression of potential biomarkers in the premalignant serially-transplantable, hyperplastic outgrowth (HPO) breast tissue lines to normal breast cancer cells from the same mice. Biomarkers present in the hyperplastic outgrowth (HPO) breast tissue lines but not in normal breast cells are biomarkers for premalignant breast cells.

The instant invention is also directed to a method of identifying biomarkers for breast cancer by screening both the premalignant serially-transplantable, hyperplastic outgrowth (HPO) breast tissue lines and malignant tumors arising therefrom for presence of the biomarkers. Biomarkers present in the malignant tumors but not in the premalignant serially-transplantable, hyperplastic outgrowth (HPO) breast tissue lines are biomarkers for breast cancer.

The following examples are given for the purpose of illustrating various embodiments of the invention and are not meant to limit the present invention in any fashion.

### EXAMPLE 1

Choice of Model System

In the mammary gland, cell-cell interactions are of paramount importance during development, when the mesenchyme functions as an inducer of epithelial morphogenesis, growth and differentiation. Development is hormone sensitive and alterations in hormonal conditions have profound developmental and morphogenic effects. The continued importance of cell-cell interaction into adulthood raises the possibility that emerging premalignant lesions of the mammary gland may be responsive to the surrounding stromal tissue. The ability to recapitulate fully differentiated structures and to separate and recombine epithelial and stromal compartments in vivo make the mammary gland an excellent model system.

One widely used genetically engineered mouse model of mammary tumorigenesis expresses the Polyoma Virus middle-T Antigen (PyV-mT) under the control of the Mouse Mammary Tumor Virus (MMTV) Long Terminal Repeat (LTR) promoter. The Polyoma Virus middle T (PyV-mT) model is of useful because the transgene rapidly produces a typical mammary hyperplasias followed by metastatic adenocarcinomas (18). Thus, the strengths of the PyV-mT model include reliable and relatively rapid tumor induction and a high rate of pulmonary metastasis (18).

Cell transformation by PyV-mT shares signal transduction pathways with the Her-2/neu gene product (11), including the PI3 kinase pathway. Her-2/neu is over-expressed in 20-30% of human breast cancers and inversely correlated with survival (26, 27). Since PyV-mT triggers many of the same pathways as erbB2 with converging signal transduction at the PI(3)′ kinase activation site and has much more rapid tumor kinetics, it has been used as a surrogate for erbB and other receptor tyrosine kinases. The PyV-mT develops mammary tumors that resemble the papillary and scirrhous carcinomas of humans (18, 28).

Although identified and described, the premalignant lesions were not biologically characterized by transplantation. However, interactions of the PyV-mT transgenics with a large number of knockouts and other transgenes have defined many of the pathways that are required for neoplastic progression. The other is the cerbB2 transgene (29) also under MMTV control, which involves the same pathway with much slower tumor development. Mammary tumorigenesis in general, and these strains in particular have several very important and useful features. Both offer these additional attributes: systems offer models to test gene effects on multistep carcinogenesis; the tumors induced by the expression of the oncogenes are adenocarcinomas that closely resemble human breast adenocarcinoma; the mammary gland undergoes almost all development after birth, at puberty and pregnancy; and, Transplantation studies are feasible, informative and required to precisely define the biological potential of the tissue and HPO lines generated.

FVB mice, expressing PyV-mT antigen under control of the mammary specific MMTV-LTR promoter, were provided by William J. Muller (McMaster University, Hamilton, Ontario, Canada). All mice were bred and maintained in a UCSD Vivarium according to NIH guidelines and were sacrificed by CO₂ inhalation.

TABLE 1
Comparison of Mammary Tumors Resulting from Transformation
with PyV-mT and cErbB2

| | | |
|---|---|---|
| Feature | PyV-mT | cErbB2 |

| | | |
|---|---|---|
| Multifocal Mammary Tumors | 80 days | 4-6 months |
| (100% female mice) | | |
| Epithelial hyperplasia, dysplasia, | 21-26 days | 2-6 months |
| Metastasis to Lung | 95 days | 6-12 months |
| | >95% | 50% |

### EXAMPLE 2

Whole Mounts and Immunohistochemistry

Mammary whole mounts were prepared as described (30). For microscopic analyses, glands fixed overnight in formalin, paraformaldehyde, or Bouin's reagent, were paraffin embedded and sectioned to 5 μm. Hematoxylin & eosin staining and appropriate immunohistochemistry or in situ hybridization was performed. Antigen retrieval was accomplished by microwaving in citrate buffer. Immunohistochemistry studies used anti-CK-8 (PH19211.xs, Binding Site, 1:300), anti-CD14 (PH504 Binding Site, 1:200), anti-SMA (A-2547 Sigma, 1:1000), anti-laminin (L-9393 Sigma, 1:1000), anti-ER_ (SC542, Santa Cruz 1:1000), anti-OPN (AF808 R&D Systems, 1:800), anti-WAP (Dr. Lothar Henninghausen, NIH, 1:4000) (31), anti-p53 (PH507.xS Binding Site, 1:1000), anti-Ki-67 (CNLki67p Novo Castro, 1:1800), anti-CD31 (SC-1506 Santa Cruz, 1:200), anti-Polyoma Virus T-ag (Ab-4, Oncogene Research, 1:15), and anti-PGr (A0098, DAKO 1:1800) (32). Anti-SMA was amplified and detected using ARK (Dako). Other antibodies were amplified and detected using the Vector ABC kit. A digoxiginated PyV-mT anti-sense probe, (Dr Robert Oshima, Burnham Institute), was detected as a blue signal using anti-digoxigen coupled alkaline phosphatase (33). Images were captured using a Kontronic camera model 8102 on an Olympus BH2 microscope, digitized using Photoshop 6.0 with the Kontron ProgRes “plug-in” module, color enhanced, balanced for contrast and printed using a Kodak 8650 dye sublimation printer. Images of nuclei were captured with a Kontron digital camera and analyzed using Image Pro Plus 4.1 (Media Cybernetics), internal programs, and statistical packages.

### EXAMPLE 3

Primary Lesion Transplantation

Donor tissue from zones 3 and 4 of inguinal mammary glands of 8 week-old PyV-mT^{+/-} females was isolated from mice anesthetized with i.p. injections of xylazine/ketamine. 1 mm³ tissue segments were transplanted into inguinal mammary fat pads of 3 week-old non-transgenic FVB females that were either cleared of epithelium as described (34, 35), or left with intact growing epithelium.

### EXAMPLE 4

Generation and Characterization of Serially-Transplantable, Hyperplastic Outgrowth Lines

Specific mammary gland lesions from zone 4 of an 8 week-old female PyV-mT^{+/-} mouse were visualized under a dissecting scope, and 1 mm³ tissue segments were surgically isolated and transplanted into cleared mammary fat pads as described above. Recipients were monitored weekly for palpable tumors. At 6-12 week intervals, non-malignant tissue was selected and retransplanted into cleared mammary fat pads and also subcutaneously at intervals to test malignant potential. At the 4^{th} generation, each of five established hyperplastic outgrowth (HPO) lines were transplanted bilaterally into 10-11 gland-cleared recipient fat pads, monitored weekly for palpable tumors and fat pads bearing palpable tumors were surgically removed. At 14 weeks and 21 weeks, all surviving mice were surgically examined under a dissecting microscope for evidence of malignancy. If no tumor was evident, weekly examination resumed. Twenty tumors emerging from hyperplastic outgrowth tissues were subcutaneously transplanted to test malignant status. Lungs were examined grossly at autopsy for evidence of metastasis.

### EXAMPLE 5

Angiogenesis

Anesthetized mice were exsanguinated with whole body PBS perfusion using a flow pump (Control Co., TX), and without pause, reperfused with Black India Ink (Sanford No 4418, 1:10 in PBS). Fat pads were prepared as whole mounts (36). Selected tissues were embedded in paraffin, sectioned, and counterstained with eosin. For anti-CD31 staining, 5 μM frozen sections were fixed in 2% formalin and stained with anti-CD31 (01951A, Pharmingen) at 1:200. Magnified 20× images (LEICA DMLB microscope) were captured with a SPOT RT digital camera (Diagnostic Instruments), imported into Photoshop 6.0 (Adobe, Inc.), and analyzed as described (35) except the data were normalized to the epithelial area.

### EXAMPLE 6

Development of Focal Atypical Lesions

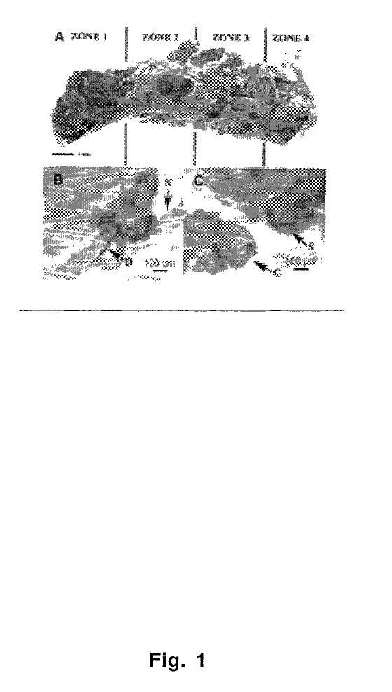

PyV-mT transgenic female mice exhibited palpable tumors beneath the nipple of several mammary glands by the 5^{th} week of life (37). Multifocal areas of mammary hyperplasia with atypia were previously described in the PyV-mT gland (18). To study these hyperplasias, whole mount preparations of inguinal mammary glands from virgin females were analyzed. For descriptive purposes, mammary glands were divided into four topographical zones (FIG. 1).

Regardless of age, a gradation of focal a typical lesions was always apparent. The advancing ductal tree appeared normal while the older, more proximal ducts exhibited hyperplastic foci. Zones 1 and 2 generally had more and larger foci than zones 3 and 4. However, by 9 weeks the entire gland sometimes had too many coalescing cellular masses to count. By 13 weeks, metastatic lesions were found in 80% (12/15) of their lungs. A majority of the small lesions visible in zones 3 and 4 were cystic out-croppings arising along or at the ends of ducts (FIG. 1B). Most were lined by a thickened, papillary epithelium that had smooth outlines, suggesting non-invasive growth (FIG. 1C, Arrow C). In contrast, Zone 1 lesions from 35 day-old female mice were frequently solid masses of cells that invaded the surrounding fat nerve sheaths. These lesions fit the morphological criteria for malignant neoplasms. In older animals, larger lesions were also present in zones 3 and 4. These lesions were frequently solid (FIG. 1C, Arrow S).

### EXAMPLE 7

Microscopic and Gene Expression Analysis

To identify the cell types present in the focal lesions and document their relative differentiation, inguinal mammary glands of 3, 5, 7, and 9-week PyV-mT mice were assessed by a combination of microscopic, morphometric, and immunohistochemical analyses. To establish a baseline for comparison with other transgenic models as well as human breast disease, a modification of the recommended immunohistochemical panel was used (6). The antibodies were chosen to yield information about 1) transgene expression, 2) cytoplasmic differentiation, 3) cell proliferation, 4) nuclear characteristics and 5) non-epithelial components including basement membrane and microvasculature. Cytokeratin 8 (CK-8) is a differentiation marker used to identify epithelial cells (38). Whey acidic protein (WAP) (39) and osteopontin (OPN) (40) are proteins expressed by differentiated secretory mammary epithelial cells. Several proteins commonly used in the diagnosis, grading and prognosis of human breast cancer were included. For example, expression of the nuclear antigen Ki-67 is commonly assessed in both humans and mice to identify proliferating cells (41-43). Estrogen receptor (ER), progesterone receptor (PgR) and p53 expression are routinely assessed in humans since they are important prognostic and predictive factors in human breast disease (43-45). Table 2 summarizes the results of the immunohistochemical stains. Several pertinent findings are highlighted in the text below.

TABLE 2
Tabulation of Marker Antigens as Determined by
Immunohistochemical Staining

| | | Terminal | Prelactating | Dysplasia | |
|---|---|---|---|---|---|
| | Duct | End Bud | Acinus | (MIN) | Tumor |
| CK-8 | 4 | 4 | 4 | 3 | var |
| CK-14 | 1 | 1 | 1 | 2 | 2 |
| WAP | ± | — | 4 | 1 | — |
| OPN | — | — | 4 | 2 | 2 |
| SMA | 1 | — | 1 | — | — |
| PgR | 3 | 4 | 3 | — | — |
| ER | 1 | 2 | 2 | 2 | var |
| p53 | — | — | — | — | ± |
| Ki-67 | 4 | 4 | 2 | 4 | 4 |
| PyV-mt | — | — | — | 2 | 4 |
| c-erb-B2 | — | 1 | 2 | var | var |
| CD-31 | — | — | — | var | — |
| Laminin | 1 | 1 | 4 | 1 | var |

Immunohistochemical stains were performed to detect the presence of selected antigens as noted. The relative intensities are recorded in a 4+/4+ scoring system based upon the intensity in a positive control slide for indicted tissues from non-transgenic FVB mammary glands (not bold) and from PyV-mT induced MIN and tumors (results in bold). Var.=variable intensity.

### EXAMPLE 8

Mammary Epithelium: Morphologic Heterogeneity

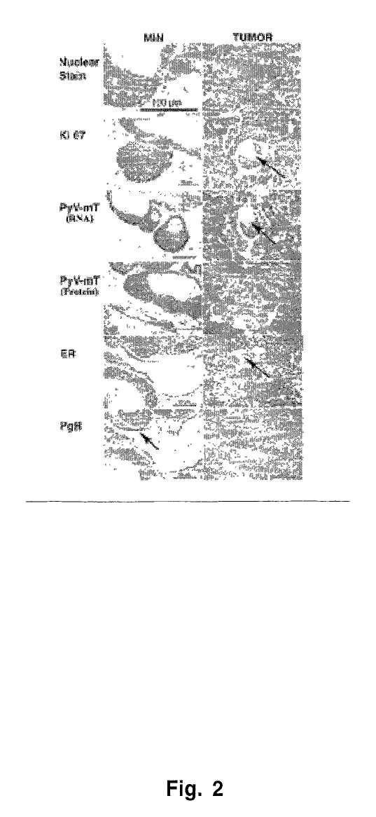

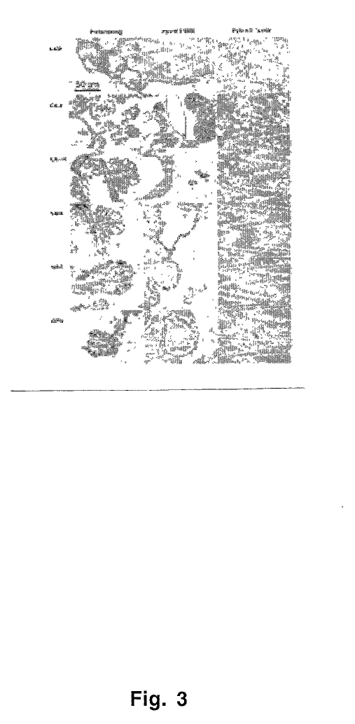

Non-invasive focal lesions were well developed by 5 weeks and could be classified into 4 subsets: simple, solid, cystic, and “mixed” solid and cystic (FIG. 1). Simple lesions were observed microscopically as semi-cystic bulges along the ducts containing one to several layers of a typical cells (FIGS. 2 and 3). Occasionally, these lesions had small gland-like structures with secondary lumens that were positive for whey acidic protein and OPN, indicating mammary secretory epithelial differentiation (FIG. 3). The configuration and staining characteristics of these lesions suggest that they may be abortive side buds.

Solid lesions were generally larger foci containing solid masses of a typical cells organized in nodular sheets. These lesions also contained osteopontin positive cells. However, they did not contain central fluid-filled spaces. Cystic lesions varied in size and complexity (FIGS. 2 and 3). Most were lined by a multi-layered epithelium that was frequently papillary and contained whey acidic protein and osteopontin positive cells (FIG. 3). These lesions were uniformly associated with significant amounts of clear fluid containing both whey acidic protein and osteopontin (FIG. 3). Some cystic lesions were directly adjacent to or connected to one or more solid cell masses and were therefore classified as “mixed” solid and cystic.

Regardless of morphology, the hyperplastic foci contained a peripheral, multi-layered rim of solid cells that expressed higher levels of PyV-mT. Interestingly, the pattern of Ki-67 positive, proliferating cells was similar to the pattern of transgene expression (FIG. 2).

### EXAMPLE 9

Myoepithelial Changes

Smooth muscle actin (SMA) and cytokeratin 14 (CK-14) expression were used to identify the myoepithelial cells surrounding normal mammary epithelium (46, 47). However, cytokeratin-14 is also expressed in some proliferating tumor cells (48). Without exception, a typical foci lacked detectable myoepithelium as measured by smooth muscle actin and cytokeratin-14 (FIG. 3). Consistent with the hypothesis that cytokeratin-14 is a proliferation cytokeratin, some cytokeratin-14 positive cells were found scattered in the more solid lesions and tumors (FIG. 3)

### EXAMPLE 10

Basement Membrane

Laminin expression, used to identify the basement membrane surrounding non-invasive tissues, was reduced but detectable at the expansile margins of a typical foci (FIG. 3). The presence of laminin suggests that the lesions were in situ, restrained by a basement membrane. In contrast, laminin stains did not reveal basement membranes around tumor cells (FIG. 3).

### EXAMPLE 11

Nuclear Changes

Nuclear characteristics were used to describe and grade mammary lesions (49). Both AgNOR and Feulgen stains, as well as analysis by flow cytometry, suggested that a typical hyperplastic lesions were primarily composed of diploid cells. The tumor cells analyzed by flow cytometry were also primarily diploid (data not shown). Focal PyV-mT-induced lesions generally contained relatively large nuclei with a coarse chromatin pattern and somewhat irregular nuclear outlines (FIGS. 2 and 3). Some overlap in morphometric parameters was noted when these nuclei were measured and compared with nuclei in normal or malignant areas in the same tissue section. However, morphometric measurements were consistent with the trend towards increased size and pleomorphism observed by the pathologist. For example, the modal (mean) area of normal, hyperplastic, and tumor nuclei is 24.9 (23.0) μm², 26.8 (30.0) μm² and 39 (35.9) μm², respectively. Further, when compared to the nuclei of proliferating acinar cells in prelactating glands of non-transgenic mice, nuclei in PyV-mT-induced a typical lesions had greater size variation and a higher proportion of Ki-67 positive cells.

### EXAMPLE 12

Vascularity

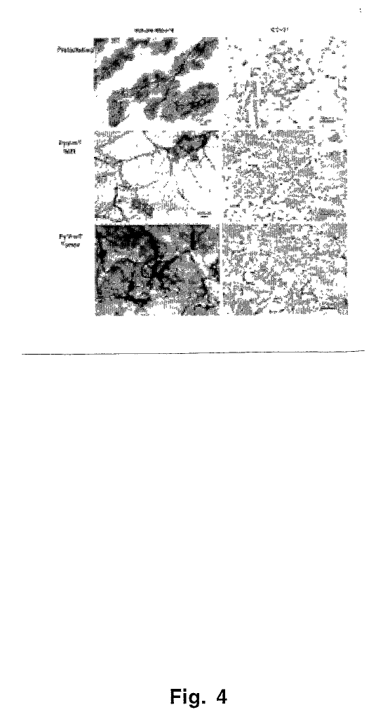

Blood vessels associated with a typical foci had larger diameters and displayed more irregular, tortuous courses (FIG. 4). In contrast, pregnancy-induced epithelial hyperplasia in non-transgenic mice is characterized by an evenly distributed network of fine vasculature (FIG. 4). The microvascular density (MVD), expressed as number of vessels per 0.45 mm squared microscopic field in non-transgenic prelactating mammary glands (89.8±8.2) was higher than that of PyV-mT hyperplastic lesions (47.1±6.4) or tumors (55.2±1.6). Vessels associated with tumors were also larger in diameter and more tortuous than either prelactating or PyV-mT-induced hyperplasia.

Each group analyzed also differs in the average amount of epithelium filling the microscopic field. Interestingly, when normalized per 0.45 mm squared of epithelium, the adjusted MVD of prelactating mammary glands (256.8±21.8) was greater than that of hyperplasias (102.5±12.8) or tumors (63.0+2.0). These data suggest that despite evidence of angiogenesis, the a typical hyperplasias and especially the tumors are not adequately perfused. This interpretation was supported by the presence of necrotic areas within some tumors.

### EXAMPLE 13

Estrogen/Progesterone Receptor and p53 Expression

Estrogen receptor (ER) and progesterone receptor (PgR) expression are important prognostic and predictive factors in human breast disease (43-45) although they have not been systematically documented in mouse models. Atypical lesions had low levels of detectable estrogen receptor (FIG. 2, Table 2). There were, however, some patches of estrogen receptor negative cells within the lesions. Progesterone receptor was detected in the ducts and acini of prelactating tissue as well as the ducts and terminal end buds of normal virgin mammary tissue (Table 2). However, no progesterone receptor positive cells were found within a typical lesions or tumors. Immunohistochemical screening for the tumor suppressor p53 is routinely assessed in humans because mutations in p53 are a strong indicator of prognosis (50). Many p53 mutations result in a nonfunctional but more stable protein that accumulates to levels detectable by immunohistochemical methods. Thus, measuring p53 protein expression by immunohistochemistry is a relatively accurate surrogate method for detecting p53 mutations (43). No evidence of p53 staining was observed in the PyV-mT-induced hyperplasias and was found in only one of twenty tumors.

### EXAMPLE 14

Characteristics of Tumors

Tumors in PyV-mT mammary glands were poorly differentiated as previously described (18) and were frequently solid masses. Ki-67 positive proliferative cells were scattered throughout the mass (FIG. 2). Some tumor cells expressed high levels of PyV-mT but the stain intensity was quite variable (FIG. 2 and Table 2). Similar to the a typical hyperplastic foci described above, tumors lacked organized myoepithelium (FIG. 3). Further, OPN positive cells were frequently scattered throughout the mass (FIG. 3), demonstrating that some cells retained secretory potential. In contrast to the a typical hyperplastic foci, tumors lacked detectable WAP (FIG. 3). Tumors also lacked organized basement membrane, suggesting invasive behavior.

### EXAMPLE 15

Transplantation Studies

To determine whether the focal a typical hyperplasias described above were malignant, the hyperplasias were subjected to “test-by-transplantation” experiments. These experiments are the standard for testing the biological potential of putative precursor lesions in murine mammary tumor models (6). Malignant tissue will grow when transplanted to a location ectopic to the mammary fat pad. In contrast, non-malignant mammary tissue will not grow outside of the mammary fat pad (5).

### EXAMPLE 16

Properties of Primary Outgrowths

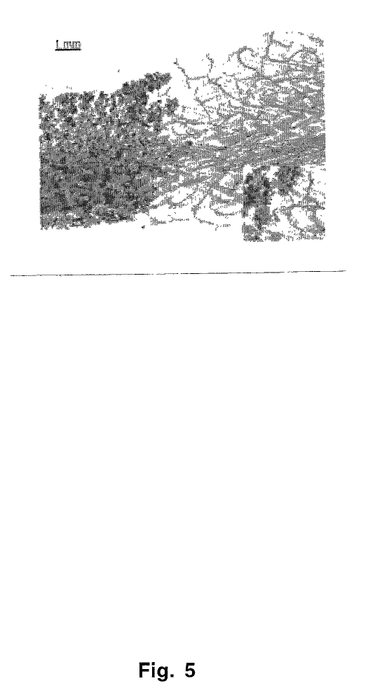

Because palpable tumors were frequently located beneath the nipple, it might be easier to identify, isolate and test putative premalignant tissue from zones distal to the nipple. Hence, foci isolated from zones 3 and 4 of 8 week-old transgenic mammary glands were transplanted both subcutaneously (ectopic) and into gland-cleared fat pads (orthotopic) of syngeneic non-transgenic mice. Whole mount analysis revealed non-invasive hyperplastic outgrowths in 64% (18/24) of transplant-recipient fat pads (FIG. 5). In contrast, no (0/14) tissues transplanted subcutaneously formed palpable tumors within a 10 to 17 week post-transplant window.

These findings suggest that the tissues isolated and transplanted from zones 3 and 4 were hyperplastic but not malignant. Fifteen primary PyV-mT outgrowths were examined microscopically. They consisted primarily of cystic masses lined by multilayered, a typical epithelium with large, pleomorphic nuclei. However, some regions contained microscopic foci of solid, invasive tumors. These tumors arose in direct continuity with hyperplastic tissue and appeared to be emerging subsets of the original transplant population. The presence of focal tumors within some outgrowths suggested that the transplanted cells were, indeed, premalignant.

### EXAMPLE 17

Growth Regulation

The growth properties of these premalignant foci were further tested by isolating and transplanting zone 4 foci into intact (non-cleared), inguinal glands of 4 week-old mice. After 10 weeks, the growing ducts and the transplants converged near the lymph node (FIG. 5). The ducts and transplanted tissues stopped growing without overlapping. The transplanted transgenic mammary outgrowths displayed the expected, non-invasive, hyperplastic morphology. The host, non-transgenic epithelium had no subgross or microscopic evidence of hyperplasia. These results indicate that the transplanted hyperplasias obey normal tissue regulation and that transgene expression does not exert a paracrine influence over the normal gland (FIG. 5). Failure of transplanted foci to grow subcutaneously or overgrow the host mammary gland confirms that their growth was restricted to the mammary fat pad and the cells were not malignant.

### EXAMPLE 18

Premalignant Serially-Transplantable, Hyperplastic, Outgrowth (HPO) Breast Tissue Lines

The biology of zone 4 hyperplastic lesions was more thoroughly studied by establishing 5 different HPO lines (Table 3). These lines are currently in the 10^{th} transplant generation. Their growth, without regression, documents that they were immortalized (7). Their malignant potential was tested by subcutaneously transplanting HPO tissue into syngeneic recipient animals. These ectopic (subcutaneous) transplants did not progress to tumors, providing strong evidence they were not malignant. However, all lines gave rise to areas of a typical hyperplasia and eventually developed tumors within the recipient fad pad, indicating that the HPO lines represent premalignant tissues. The tumors that developed within these HPOs grew in ectopic sites and metastasized, confirming that they were malignant.

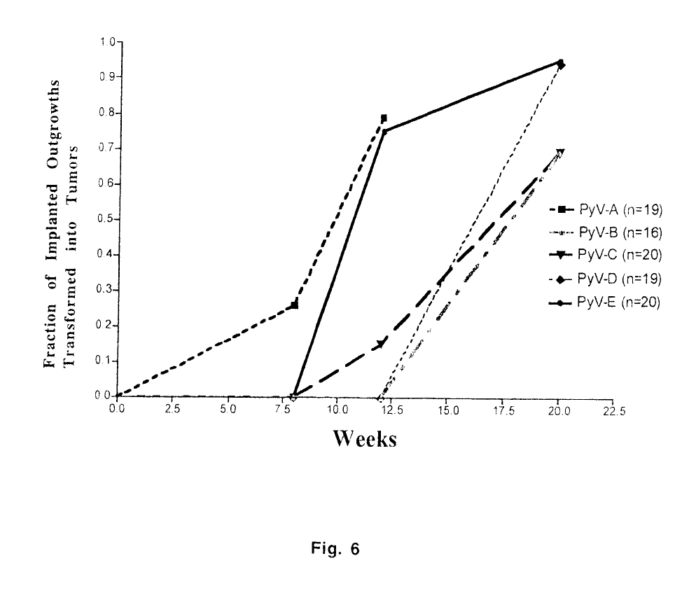

The subgross morphology of the five serially-transplantable, hyperplastic outgrowth lines are not identical, but all lines gave rise to areas of a typical lobular hyperplasia that eventually develop tumors, suggesting that these hyperplastic outgrowths are indeed premalignant tissues as shown in Table 3. However, their malignant potential is quite variable. For example, the incidence and latency of tumor development differs. Two lines have a high incidence of malignant transformation after 14 weeks and the other three have a low incidence (FIG. 6). The malignant potential of tumors arising within outgrowths in these lines was proven by the rapid, invasive growth of 6 of 7 transplants into subcutaneous tissue. Additionally, pulmonary metastases have been found in the lungs of mice bearing tumors arising from 4 of 5 transplant lines. The difference in tumor latencies implies heterogeneity among the premalignant lesions that arise within the PyV-mT mammary gland.

TABLE 3
Tumor Incidence and Morphology of Serially-transplantable,
hyperplastic outgrowth lines

| | Tumor Incidence (Fat | | |
|---|---|---|---|
| | Pads with Tumors/ | | |
| HPO | Total Transplanted | Time | Morphological |
| Line | Fat Pads) (%) | (Weeks) | Characteristics |
| A | 14/20 (70) | 14 | cystic/LA |
| B | 0/17 (0) | 14 | LA/cystic |
| | 11/17 (64.7) | 21 | |
| C | 3/20 (15) | 14 | cystic/LA |
| | 11/18 (61.1) | 21 | |
| D | 0/18 (0) | 14 | LA |
| | 13/18 (72.2) | 21 | |
| E | 14/20 (70) | 14 | LA |
| | 18/20 (90) | 21 | |

Zone 4 lesions from 9 week-old mice were serially transplanted to 4-week-old gland cleared recipients. After 4 passages, the tumor incidence for each of the five HPO Lines was measured at 14 and 21 weeks and is reported as the number of fat pads with tumor(s)/total (number) of fat pads with transplants. HPO morphological characteristics are reported as cystic, LA (lobuloalveolar) or both.

### EXAMPLE 19

Generation of Serially-Transplantable, Hyperplastic Outgrowth Breast Tissue Lines From Zone 1

Experiments have been initiated to assess the biological potential of the foci located in Zone 1 near the nipple, where tumors form quite early. Outgrowths were established from Zone 1, and in the same manner as the Zone 4 outgrowths were isolated with the exception that the donor animals were only 4 weeks old in this case. Comparison of the growth of Zone 1 and Zone 4 primary transplants is shown in Table 4. This data clearly show that there is a difference in the biological potential between the Zone 1 and Zone 4 cells.

TABLE 4
Comparison of Transplants from Zone 1 Hyperplastic Outgrowths
with Transplants from Zone 4 hyperplastic Outgrowths

| Transplanted | Subcutaneous | |
|---|---|---|
| Tissue | Growth | Growth in Fat Pad |
| Zone 1 | 6/8 | 12/12 |
| Zone 4 | 0/15 | 23/28 |

The analysis of Zone 1 outgrowth mammary tissues was conducted and assessed in preliminary studies similar to those for Zone 4 lesions. Analysis of the latency period (time to develop palpable tumor) indicated the stage of the cells in transformation. The experiments used wild type host mice so that tumor, hyperplasia, and premalignant lesions can be assessed to evaluate the stepwise transformation to adenocarcinoma. The histology and immunohistochemistry of the Zone 1 outgrowth cells is analyzed to provide established diagnosis criterion for premalignancy and malignancy.

Discussion

Genetically engineered mouse models have become popular in the field of cancer research because they give researchers the unique opportunity to investigate the multiple aspects of cancer within an intact organism. Analysis of these mice has already provided insights into mechanisms of oncogenesis and metastasis (16, 17, 51). However, basic studies of the biology and pathology of premalignant lesions have generally lacked mammary transplantation experiments that provide important information about their growth potential (5, 6). Since early detection and intervention in human breast disease are critical to cancer prevention, the attributes of putative precursor lesions in model systems need to be carefully characterized and validated by direct comparison with human lesions. Here, a comprehensive characterization of premalignant PyV-mT mammary lesions was provided, including mammary transplantation experiments and immunohistochemical stains commonly used in both mouse and human histopathology. These studies will provide a basis for direct comparisons with other models and with human breast disease (6).

The similarities between PyV-mT premalignant lesions and many types of human a typical hyperplasias emphasize the value of this model system. Like their human counterparts, they were morphologically heterogeneous with highly proliferative focal areas containing a typical nuclei. They had abnormal microvasculature, lacked an organized myoepithelium, and remained within an intact basement membrane. Further, they expressed differentiation markers that were not expressed in tumors. Although they commonly expressed ER, they never expressed detectable PgR. The accumulation of p53 was seldom detected. It is noteworthy that PyV-mT and Ki-67 expression were concentrated at the periphery of MIN lesions, suggesting that transgene expression stimulated growth.

Transgenic tumors were distinct from mammary intraepithelial neoplasia lesions in several respects. The tumors were less differentiated than mammary intraepithelial neoplasia lesions, they had larger, more pleomorphic nuclei; they lacked a basement membrane, had increasingly abnormal vasculature, and were frequently metastatic. PyV-mT expression was variable in the tumors, suggesting that transgene expression is important for initiation but not maintenance of the malignant phenotype.

The results of the test-by-transplantation experiments underscore the value of these experiments in the characterization of mammary intraepithelial neoplasia in mouse models. The early onset of palpable mammary masses supported the hypothesis that expression of PyV-mT transgene alone might be sufficient for tumorigenesis and, therefore, these mice represented a single-step model of transformation (18). The biological studies described herein have proven tumorigenesis is, indeed, a multi-step process in this model. A similar literature controversy exists regarding whether transgenic MMTV/activated neu mice are a model for single step or multi-step neoplastic progression (52, 53). Biological studies similar to those described here would likely resolve this issue.

The HPO lines produced in this study represent a new tool for the study of intermediate stages of PyV-mT-induced malignant progression. Several characteristics of these lines make them ideal for the detailed studies required to define the molecular pathways necessary to develop a malignant phenotype. The HPO lines produced in this study have stable and documented predictable growth behavior. The difference in the tumor latencies of the lines implies heterogeneity among the PyV-mT-induced premalignant hyperplasias. Likewise, human breast hyperplasias are associated with variable malignancy risk. The HPO lines make it possible to isolate and compare premalignant tissue and the tumors arising within the same tissue. These lines will facilitate investigations that focus on specific molecular changes related to malignancy, such as signaling-pathway alterations during malignant progression. The PyV-mT transgene activates the Erb-B2/PI3 kinase pathway that is frequently dysregulated in human breast cancers (36). The HPO lines thereby enhance the utility of the PyV-mT transgenic model by making accessible the full range of events occurring in multi-step mammary tumorigenesis, from the inception of the lesion to the development of pulmonary metastasis.

Meaningful information needed to accurately predict the biological outcome of hyperplastic lesions in humans will be gained by integrating molecular data with pathobiological findings, that together provide insight into the behavior of premalignant tissues as well as their response to therapeutics (5). Since investigations of the biological potential of human a typical hyperplasias are limited to retrospective epidemiological analysis, mouse models of mammary intraepithelial neoplasia are particularly valuable because they are amenable to prospective “test-by-transplantation.” More attention to the biological growth properties of premalignant lesions in these systems will provide essential clues for unraveling the mysteries of tumor progression.

The following references were cited herein:

1. Page, et al., Breast Cancer Res Treat. 51: 195-208, 1998.

2. Simpson, et al., Seminars in Oncology. 23: 428-35, 1996.

3. Fitzgibbons, et al., Prognostic factors in breast cancer. College of American Pathologists Consensus Statement 1999, Archives of Pathology and Laboratory Medicine. 124: 966-78, 2000.

4. Medina, D., Journal of Mammary Gland Biology and Neoplasia. 1: 5-19, 1996.

5. Cardiff, et al., Journal of Mammary Gland Biology and Neoplasia. 5, 2000.

6. Cardiff, et al., Oncogene. 19: 968-988, 2000.

7. Cardiff, R., Adv Cancer Res. 42: 167-90, 1984.

8. Cardiff, R. D. 1998. Journal of Mammary Gland Biology and Neoplasia. 3:3-5, 1998.

9. Cardiff, R. D., and R. J. Munn 1998. The Histopathology of Transgenes and Knockouts in the Mammary gland., p. 177-202. In G. Heppner (ed.), Breast Cancer: Advances in Oncobiology. JAI Press Inc., 1998.

10. Amundadottir, et al., Breast Cancer Res Treat. 39: 119-35, 1996.

11. Dankort, D. and Muller, W. Oncogene. 19: 1038-1044, 2000.

12. Siegel, et al., Bioessays. 22: 554-63, 2000.

13. Adams, J., and S. Cory Science. 254:1161-7,1991.

14. Christofori, et al., Semin Cancer Biol. 5:3-12, 1994.

15. Dankort, et al., Cancer Treatment and Research. 83:71-88, 1996.

16. Hanahan, D. Transgenic mice as probes into complex systems, Science. 246: 1265-75, 1989.

17. Webster, et al., Semin Cancer Biol. 5: 69-76, 1994.

18. Guy, et al., Mol Cell Biol. 12: 954-61, 1992.

19. Cardiff, R. D. The Biology of Mammary Transgenes: Five Rules., J. Mammary Gland Biology and Neoplasia. 1: 61-73, 1996.

20. Cardiff, et al., Journal of Mammary Gland Biology and Neoplasia. 5:421-437, 2000.

21. Cardiff, et al., Anticancer Res, 1988. 8(5A): p. 925-33.

22. Cardiff, R. D., Adv Cancer Res, 1984. 42: p. 167-90.

23. Cardiff, et al., Cancer, 1977. 39(6 Suppl): p. 2734-46.

24. Medina, D., The preneoplastic state in mouse mammary tumorigenesis. Carcinogenesis, 1988. 9(7): p. 1113-9.

25. Medina, D., Preneoplasia in mammary tumorigenesis. Cancer Treatment and Research, 1996. 83: p. 37-69.

26. Clark, et al., Cancer Research. 51:944-8, 1991.

27. Slamon, et al., Science. 235:177-82, 1987.

28. Guy, et al., Genes Dev, 1994. 8(1): p. 23-32.

29. Guy, et al., Proc Natl Acad Sci U S A, 1992. 89(22): p. 10578-82.

30. Rasmussen, et al., Preparing Mammary Gland Whole Mounts from Mice. In: E. M. M. I. a. B. B. Asch. (ed.) Methods in Mammary Gland Biology and Breast Cancer Research., pp. 75-85. NY, Boston, Dordrecht, London, Moscow.: Kluwer Academic/Plenum Publishers, 2000.

31. Wall, et al., et al., Proc Natl Acad Sci U S A. 88: 1696-700., 1991.

32. Shyamala, et al., Journal of Steroid Biochemistry and Molecular Biology. 63: 251-9, 1997.

33. Hirsch, et al., et al., Journal of Virology. 69: 955-967, 1995.

34. Young, L. J. T. The cleared mammary fat pad and the transplantation of mammary gland morphological structures and cells. In: M. M. Ip and B. B. Asch. (eds.), Methods in Mammary Gland Biology and Breast Cancer Research., pp. 67-74. NY, Boston, Dordrecht, London, Moscow.: Kluwer Academic/Plenum Publishers, 2000.

35. DeOme, et al., Cancer Research. 19: 515-525, 1959.

36. Gershtein, et al., Clin Chim Acta. 287: 59-67., 1999.

37. Neznanov, et al., Cancer Res. 59: 4242-6, 1999.

38. Brotherick, et al., Cytometry. 32: 301-8., 1998.

39. Hennighausen, et al., Cell Growth Differ. 5: 607-13, 1994.

40. Brown, et al., Molecular Biology of the Cell. 3: 1169-1180, 1992.

41. Pierga, et al., Breast Cancer Research and Treatment. 37: 57-64, 1996.

42. Brown, et al., Clinical Cancer Research. 2: 585-92, 1996.

43. Allred, et al., Modern Pathology. 11: 155-68, 1998.

44. Parl, et al., Cancer. 54: 2237-42, 1984.

45. Reiner, et al., Cancer Research. 50: 7057-61, 1990.

46. Jarasch, et al., Hum Pathol. 19: 276-89., 1988.

47. Pellegrino, et al., Cancer Res. 48: 5831-6., 1988.

48. Smith, et al., Cell Growth and Differentiation. 4: 161-70, 1990.

49. Page, et al., Modern Pathology. 11: 120-8, 1998.

50. Allred, et al., Breast Cancer Res Treat. 32: 13-8, 1994.

51. Pattengale, et al., Am J Pathol. 135: 39-61, 1989.

52. Muller, et al., Cell. 54: 105-15, 1988.

53. Bouchard, et al., Cell. 57: 931-6, 1989.

54. Di Carlo, et al., Lab Invest. 79: 1261-9, 1999.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method of isolating premalignant, serially-transplantable, hyperplastic outgrowth breast tissue lines from mammary gland tissue, comprising the steps of:
isolating hyperplastic foci from regions of mammary gland tissue in transgenic mice expressing a transgene that genetically predisposes said mice to develop breast cancer;
implanting cells from said foci both subcutaneously and in the inguinal mammary fat pads of syngeneic, non-transgenic mice;
identifying foci which grow in said fat pads but not subcutaneously as premalignant, hyperplastic outgrowths; and,
establishing clonal serially-transplantable breast tissue lines from said outgrowths.

2. The method of claim 1, wherein said hyperplastic foci are isolated from regions of mammary gland tissue distal from or proximal to the nipples of said mice.

3. The method of claim 1, wherein said hyperplastic foci are isolated from mammary gland tissue of said transgenic mice before tumor development occurs.

4. A premalignant, serially-transplantable, hyperplastic outgrowth (HPO) breast tissue line isolated by the method of claim 1.

5. A method of isolating premalignant, serially-transplantable, hyperplastic outgrowth breast tissue lines from mammary gland tissue, comprising the steps of:
isolating hyperplastic foci from regions of mammary gland tissue in PyV-mt transgenic mice;
implanting cells from said foci both subcutaneously and in the inguinal mammary fat pads of syngeneic, non-transgenic mice;
identifying foci which grow in said fat pads but not subcutaneously as premalignant, hyperplastic outgrowths; and,
establishing clonal serially-transplantable breast tissue lines from said outgrowths.

6. The method of claim 5, wherein said hyperplastic foci are isolated from mammary gland tissue of said transgenic mice before tumor development occurs.

7. The method of claim 5, wherein said hyperplastic foci are isolated from regions of mammary gland tissue distal from or proximal to the nipples of said mice.

8. A premalignant, serially-transplantable, hyperplastic outgrowth (HPO) breast tissue line isolated by the method of claim 5.

9. The premalignant, serially-transplantable, hyperplastic outgrowth breast tissue line of claim 8, wherein said tissue line is selected from the group consisting of serially-transplantable hyperplastic outgrowth breast tissue lines A, B, C, D, and E.

10. The premalignant, serially-transplantable, hyperplastic outgrowth breast tissue line of claim 8, wherein said serially-transplantable breast tissue line develops into tumors at a predicable rate with a predictable morphology when transplanted into the mammary glands or the inguinal mammary fat pads of a recipient mouse.

11 The premalignant, serially-transplantable, hyperplastic outgrowth breast tissue line of claim 10, wherein said tumors metastasize to the lungs of said recipient mouse.

12. A method of screening for chemotherapy agents which block metastasis comprising the steps of:
implanting the serially-transplantable breast tissue line of claim 8 into the inguinal mammary fat pads of syngeneic, non-transgenic recipient mice;
administering a putative chemotherapy agent to a subset of said mice; and,
analyzing the lungs of said mice for metastatic tumors, wherein if fewer metastatic tumors are observed in the lungs of mice receiving said agent compared to mice which did not receive said agent, said agent is an effective chemotherapeutic agent for blocking metastasis.

13. The method of claim 12, wherein said serially-transplantable breast tissue line is selected from the group consisting of serially-transplantable hyperplastic outgrowth breast tissue lines A, B, C, D, and E.

14. A method of screening for new chemopreventive agents against breast cancer comprising the steps of:
implanting the serially-transplantable breast tissue line of claim 8 into the inguinal mammary fat pads of syngeneic, non-transgenic recipient mice;
administering a putative chemopreventative agent to a subset of said mice; and,
analyzing said mice for the development of malignant tumors, wherein if fewer malignant tumors are observed in the subset of mice receiving said agent compared to mice which did not receive said agent, said agent is an effective chemopreventive agent against breast cancer.

15. The method of claim 14, wherein said serially-transplantable breast tissue line is selected from the group consisting of serially-transplantable hyperplastic outgrowth breast tissue lines A, B, C, D, and E.

16. A method of identifying biomarkers for premalignant breast cells, comprising the steps of:
screening the serially-transplantable breast tissue lines of claim 8 for the expression of biomarkers;
screening normal breast cells for the expression of said biomarkers; and
comparing the biomarkers expressed in said serially-transplantable breast tissue lines to the biomarkers expressed in said normal breast cells, wherein a marker expressed in said serially-transplantable breast tissue lines but not expressed in normal breast cells is a biomarker for premalignant breast cells.

17. The method of claim 16, wherein said serially-transplantable breast tissue line is selected from the group consisting of serially-transplantable hyperplastic outgrowth breast tissue lines A, B, C, D, and E.

18. The method of claim 17, wherein said screening is performed using microarray technology.

19. A method of identifying biomarkers for breast cancer cells comprising the steps of:
screening malignant tumors arising from the serially-transplantable breast tissue lines of claim 8 for the expression of biomarkers;
screening the serially-transplantable breast tissue lines of claim 8 for said biomarkers; and,
comparing the biomarkers expressed in said malignant tumors to the biomarkers expressed in said serially-transplantable breast tissue lines, wherein a biomarker expressed in the malignant tumors but not in said serially-transplantable breast tissue lines is a biomarker for breast cancer.

20. The method of claim 19, wherein said serially-transplantable breast tissue line is selected from the group consisting of serially-transplantable hyperplastic outgrowth breast tissue lines A, B, C, D, and E.

21. The method of claim 19, wherein said screening is performed using microarray technology.

22. A method of determining the effect of a gene on the development of breast cancer, comprising the steps of:
transplanting the serially-transplantable breast tissue line of claim 8 into the inguinal mammary fat pads of a recipient mice expressing said gene;
transplanting said serially-transplantable breast tissue line into the inguinal mammary fat pads of otherwise syngeneic recipient mice which lack expression of said gene; and
comparing the development of tumors in said mice, wherein differences in tumor development between mice lacking expression of said gene and mice expressing said gene are indicative of the effect of said gene on breast cancer development.
